(19) United States
(12) Patent Application Publication
(10) Pub. No.: US 2025/0120576 A1
(43) Pub. Date: Apr. 17, 2025

(54) SYSTEMS AND METHODS FOR ADJUSTING VIEWING DIRECTION

(71) Applicant: Noah Medical Corporation, San Carlos, CA (US)

(72) Inventors: Michael Herron, Sunnyvale, CA (US); Akihiro Takagi, San Mateo, CA (US); John Shen, San Jose, CA (US)

(21) Appl. No.: 18/973,406

(22) Filed: Dec. 9, 2024

Related U.S. Application Data
(63) Continuation of application No. PCT/US2023/068793, filed on Jun. 21, 2023.
(60) Provisional application No. 63/357,451, filed on Jun. 30, 2022.

(51) Int. Cl. A61B 1/00; A61B 1/045; A61B 1/05; A61M 25/00

(52) U.S. Cl. CPC A61B 1/00183; A61B 1/000095; A61B 1/00096; A61B 1/00193; A61B 1/045; A61B 1/051; A61M 25/0074

(57) ABSTRACT

A method is provided for adjusting a viewing direction for an articulatable medical device. The method comprises: receiving a command indicating adjusting a viewing direction of the articulatable medical device; determining a sensing region from an imaging sensor based at least in part on the viewing direction, the imaging sensor is located at a distal end of the articulatable medical device; generating an image based on signals from the sensing region; and processing the image to correct at least one of a projection distortion, contrast and color of the image.

### CROSS-REFERENCE

This application is a continuation of International Patent Application No. PCT/US2023/068793, filed Jun. 21, 2023, which claims priority to U.S. Provisional Patent Application No. 63/357,451, filed on Jun. 30, 2022, which is entirely incorporated herein by reference.

### BACKGROUND

Robotics technology has advantages that can be incorporated into endoscopes for a variety of applications, including bronchoscope. For example, by exploiting soft deformable structures that are capable of moving effectively through a complex environment like inside the main bronchi, one can significantly reduce pain and patient discomfort. During endoluminal endoscopic procedures, the endoluminal endoscopic system may be equipped with sensors such as electromagnetic (EM) three-dimensional (3D) sensors registering itself to the CT image or the patient anatomy. The EM information along with a direct visualization system (e.g., camera) may allow a physician to manipulate the endoscopic device to the site of the lesion and/or identify medical conditions based on the direct vision. In some cases, the image data stream may be captured using a stereoscopic camera. A physician may operate inside a subject such as a substantially circular tunnel (e.g., colon, trachea, bronchi, esophagus, etc.) by controlling a physical/mechanical orientation of the camera to have different viewing directions (line-of-sight) inside the subject. It is desirable to provide an improved and/or alternative method for adjusting a viewing direction for the endoluminal endoscopic procedures.

### SUMMARY

A need exists to improve a control of the vision system of an endoluminal endoscopic device. The endoscope system of the present disclosure may provide enhanced vision capability. In particular, the methods and systems herein may allow for adjustment of viewing directions (e.g., inside a subject) of the vision system without changing a physical/mechanical orientation of the imaging device (e.g., camera). In some embodiments, the present disclosure may provide methods and systems for digitally adjusting viewing direction (e.g., digital tilt, digital pan, etc.) during the endoluminal endoscopic procedures. In some cases, the viewing direction may be adjusted by selecting a partial readout of the imaging sensor corresponding to a viewing direction/angle. For instance, the system and method herein may receive a command indicating a desired viewing direction (e.g., tilt angle) and may determine a region (e.g., addresses of pixels) in a sensor array corresponding to the desired viewing direction for outputting sensor readout. This beneficially allows for processing/outputting only partial or portion of a full sensor array readout thereby reducing the power consumption of the vision system.

In an aspect, methods and systems are provided for digitally adjusting a viewing direction of the vision system. In some embodiments, a viewing direction may be digitally adjusted by selecting a partial readout of an imaging sensor corresponding to a viewing direction/angle. For instance, the system and method herein may receive a command indicating a desired viewing direction (e.g., tilt angle) and may determine a region (e.g., addresses of pixels) in a sensor array corresponding to the desired viewing direction for outputting sensor readout. This beneficially allows for processing/outputting only partial or portion of a full sensor array readout thereby reducing the power consumption of the vision system. In some cases, high frame readout may be performed in the selected region such that bandwidth and power consumption can be reduced.

In an aspect, a method is provided for controlling a vision system for an articulatable medical device. The method comprises: receiving a command indicative of adjusting a viewing direction of the articulatable medical device; determining a sensing region on an imaging sensor based at least in part on the viewing direction; generating an image based on signals from the sensing region; and processing the image to correct at least one of a projection distortion, a contrast and a color of the image.

In a related yet separate aspect, a non-transitory computer-readable storage medium including instructions that, when executed by one or more processors, cause the one or more processors to perform operations. The operations comprise: receiving a command indicative of adjusting a viewing direction of the articulatable medical device; determining a sensing region on an imaging sensor based at least in part on the viewing direction; generating an image based on signals from the sensing region; and processing the image to correct at least one of a projection distortion, a contrast and a color of the image.

In some embodiments, the viewing direction comprises atilt angle. In some cases, the sensing region is a portion of the imaging sensor and is selected in a vertical direction. In some embodiments, the signals are read out from the sensing region via a row selector and a column selector. In some embodiments, an array of photodiodes are enabled upon determining the sensor region and wherein photodiodes not in the sensing region are disabled. In some embodiments, the sensing region is determined based on the viewing direction and a predetermined mapping relationship.

In some embodiments, the vision system comprises two of the imaging sensors providing stereoscopic imaging. In some cases, different sensing regions in the two of the imaging sensors are selected to align the different sensing regions in a vertical direction. In some embodiments, the projection distortion, the contrast or the color of the image is corrected by applying image transformation.

In some embodiments, the method further comprises decomposing the viewing direction of the articulatable medical device into a first angle and a second angle. In some cases, the first angle is used to determine the sensing region on the imaging sensor and the second angle is used to control an orientation of the imaging sensor. In some embodiments, the imaging sensor is located at a distal end of the articulatable medical device.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of an assembly of an endoscope system, in accordance with some embodiments of the present disclosure.

FIG. 2 shows an example of a robotic endoscope, in accordance with some embodiments of the invention.

FIG. 3 shows an example of an instrument driving mechanism providing a mechanical and electrical interface to a handle portion of a robotic endoscope, in accordance with some embodiments of the invention.

FIG. 4 shows an example of a configuration of a distal portion of the system.

FIG. 5 shows another example of a distal tip of an endoscope.

FIG. 6 shows an example of distal portion of the catheter with integrated imaging device and the illumination device.

FIG. 7 schematically illustrates a method of performing digital tilt of a vision system.

FIG. 8 shows an example of complementary metal oxide semiconductor (CMOS) image sensor with digitally viewing direction adjustment capability.

FIG. 9 shows an example of a stereo camera system with digital tilt capabilities.

FIG. 10 shows an example of distortion across an entire image frame/full sensor readout.

FIG. 11 shows an example of correcting distortion.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

While exemplary embodiments will be primarily directed at an endoscope, robotic bronchoscope, or flexible instrument, one of skill in the art will appreciate that this is not intended to be limiting, and the devices described herein may be used for other therapeutic or diagnostic procedures and in other anatomical regions of a patient's body such as a digestive system, including but not limited to the esophagus, liver, stomach, colon, urinary tract, or a respiratory system, including but not limited to the bronchus, the lung, and various others.

The embodiments disclosed herein can be combined in one or more of many ways to provide improved diagnosis and therapy to a patient. The disclosed embodiments can be combined with existing methods and apparatus to provide improved diagnostic or surgical procedures, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

The methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU), graphic processing unit (GPU), or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of an endoscope or catheter may correspond to a proximal location of an elongate member of the patient, and a proximal location of the endoscope or catheter may correspond to a distal location of the elongate member of the patient.

An endoscope system as described herein, includes an elongate portion or elongate member such as a catheter. The terms “elongate member” and “catheter” are used interchangeably throughout the specification unless contexts suggest otherwise. The elongate member can be placed directly into the body lumen or a body cavity. In some embodiments, the system may further include a support apparatus such as a robotic manipulator (e.g., robotic arm) to drive, support, position or control the movements and/or operation of the elongate member. Alternatively or in addition to, the support apparatus may be a hand-held device or other control devices that may or may not include a robotic system. In some embodiments, the system may further include peripheral devices and subsystems such as imaging systems that would assist and/or facilitate the navigation of the elongate member to the target site in the body of a subject.

The endoscope system of the present disclosure may provide enhanced vision capability. In particular, the methods and systems herein may allow for adjustment of viewing directions (e.g., inside a subject) of the vision system without changing a physical/mechanical orientation of the imaging device (e.g., camera). In some embodiments, the present disclosure may provide methods and systems for digitally adjusting viewing direction (e.g., digital tilt, digital pan, etc.) during the endoluminal endoscopic procedures. In some cases, the viewing direction may be adjusted by selecting a partial readout of the imaging sensor corresponding to a viewing direction/angle. For instance, the system and method herein may receive a command indicating a desired viewing direction (e.g., tilt angle) and may determine a region (e.g., addresses of pixels) in a sensor array corresponding to the desired viewing direction for outputting sensor readout. This beneficially allows for processing/outputting only partial or portion of a full sensor array readout thereby reducing the power consumption of the vision system.

In some cases, the sensor readout may be further processed to generate an image with an image quality substantially the same as performing a mechanical tilt of the camera. In some cases, the image quality across a full image sensor may not be uniform due to the optical system physical characteristics. For example, optical factors such as distortion, contrast (e.g., modulation transfer function (MTF)), color and the like may not be uniform across the entire imaging sensor. The method and system herein may provide a uniform image quality regardless which portion/region of the image sensor the signals are read out from. For example, an output image generated based on the partial readout may be further processed such that the one or more optical parameters (e.g., MTF, distortion, color, etc.) of a final output image may be substantially uniform regardless the digital viewing direction. Details about the partial readout method and image processing method for performing the digital viewing direction adjustment are described later herein.

In some embodiments, the sensing system of the endoscopic device may comprise at least direct vision (e.g., camera). The direct vision may have a capability of digitally adjusting viewing direction as described elsewhere herein. The direct vision of the endoscopic device may have reduced power consumption without compromising performance of the system. The sensing system may also comprise positional sensing (e.g., EM sensor system, optical shape sensor, accelerometers, gyroscopic sensors), or other modalities such as ultrasound imaging.

The direct vision may be provided by an imaging device such as a camera. A camera may comprise imaging optics (e.g., lens elements) and image sensor (e.g., CMOS or CCD). The field of view of the imaging device may be illuminated by an illumination system. The imaging device may be located at the distal tip of the catheter or elongate member of the endoscope. In some cases, the direct vision system may comprise an imaging device and an illumination device. In some embodiments, the imaging device may be a video camera. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). In some cases, the image sensor may comprise an array (two-dimensional array) of optical sensors. The imaging device may be a low-cost camera that can be integrated into a tip of the endoscopic device.

In some cases, the endoscope system may incorporate a positional sensing system such as electromagnetic (EM) sensor, fiber optic sensors, and/or other sensors. For instance, the positional sensing system may be used to register the endoscope with preoperatively recorded surgical images thereby locating a distal portion of the endoscope with respect to a patient body or global reference frame. The position sensor may be a component of an EM sensor system including one or more conductive coils that may be subjected to an externally generated electromagnetic field. Each coil of EM sensor system used to implement positional sensor system then produces an induced electrical signal having characteristics that depend on the position and orientation of the coil relative to the externally generated electromagnetic field. In some cases, an EM sensor system used to implement the positional sensing system may be configured and positioned to measure at least three degrees of freedom e.g., three position coordinates X, Y, Z. Alternatively or in addition to, the EM sensor system may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

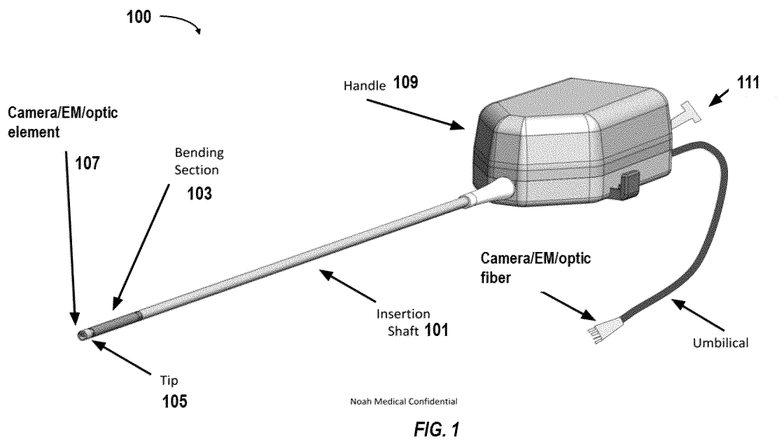

In an aspect of the invention, a flexible endoscope with improved performance at reduced cost is provided. FIG. 1 illustrates an example of a flexible endoscope 100, in accordance with some embodiments of the present disclosure. As shown in FIG. 1, the flexible endoscope 100 may comprise a handle portion 109 and a flexible elongate member to be inserted inside of a subject. In some embodiments, the flexible elongate member may comprise a shaft (e.g., insertion shaft 101), steerable tip (e.g., tip 105) and a steerable section (bending section 103). The endoscope 100 may also be referred to as steerable catheter assembly as described elsewhere herein. In some cases, the endoscope 100 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 100 may comprise a handle portion 109 that may include one or more components configured to process image data, provide power, or establish communication with other external devices. For instance, the handle portion may include a circuitry and communication elements that enables electrical communication between the steerable catheter assembly 100 and an instrument driving mechanism (not shown), and any other external system or devices. In another example, the handle portion 109 may comprise circuitry elements such as power sources for powering the electronics (e.g., camera, electromagnetic sensor) of the endoscope. In some cases, a light source assembly (including one or more laser sources) of the illumination system may be located at the handle portion. Alternatively, the light source assembly may be located at the instrument driving mechanism, the robotic support system or hand-held controller.

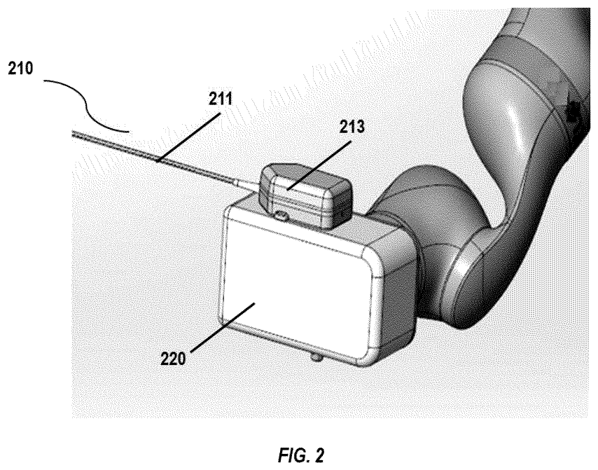

The one or more components located at the handle may be optimized such that expensive and complicated components may be allocated to the robotic support system, a hand-held controller or an instrument driving mechanism thereby reducing the cost and simplifying the design the disposable endoscope. In some cases, the handle portion may be in electrical communication with the instrument driving mechanism (e.g., FIG. 2, instrument driving mechanism 220) via an electrical interface (e.g., printed circuit board) so that image/video data and/or sensor data can be received by the communication module of the instrument driving mechanism and may be transmitted to other external devices/systems. In some cases, the electrical interface may comprise an optical interface such as connector interface for the illumination system (e.g., optic fiber connector).

In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some cases, the handle portion 109 may comprise one or more mechanical control modules such as lure 111 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 100. The mechanical interface may allow the steerable catheter assembly 100 to be releasably coupled to the instrument driving mechanism. For instance, a handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

In the illustrated example, the distal tip of the catheter or endoscope shaft is configured to be articulated/bent in two or more degrees of freedom to control the direction of the endoscope. A desired camera view or viewing direction may be controlled by articulating the distal tip of the catheter. This is also referred to as mechanically or physically adjusting a viewing direction of vision system. In some embodiments, the present disclosure provides methods and systems to digitally adjust the viewing direction in addition to or instead of controlling the camera physical orientation. Details about the methods and systems for the improved viewing direction adjustment are described later herein.

As illustrated in the example, imaging device (e.g., camera), position sensors (e.g., electromagnetic sensor), and one or more optic elements (e.g., diffractive optic element) 107 is located at the tip of the catheter or endoscope shaft 105. For example, line of sight of the camera may be controlled by controlling the articulation of the bending section 103. In some instances, the angle of the camera may be adjustable such that the line of sight can be adjusted without or in addition to articulating the distal tip of the catheter or endoscope shaft. For example, the camera may be oriented at an angle (e.g., tilt angle) with respect to the axial direction of the tip of the endoscope with aid of an optimal component. In some embodiments, a viewing direction of the camera may be digitally adjusted. In some cases, the imaging device may have capability of digitally adjusting a viewing direction (e.g., tilt angle) without or in combination with performing an articulation control of the camera. For example, upon receiving a command to tilt the view direction of the camera by a certain degrees of angle, the system may automatically decompose the tilt angle by determining a first fraction of the angle achieved via mechanical tilt of the camera (e.g., articulate the camera upwards by the first fraction) and a second fraction of the angle achieved via the digital tilt (e.g., determining a partial readout of the image sensor corresponding to the second fraction of the angle).

The distal tip 105 may be a rigid component that allow for positioning sensors such as electromagnetic (EM) sensors, imaging devices (e.g., camera) and illuminating component elements or other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5D or 6D space. This may provide a visual guide to an operator when driving the bronchoscope towards the target site. Details about the tip design and the plurality of components embedded at the tip are described later herein.

The endoscope may have a unique design in the shaft component. In some cases, the insertion shaft of the endoscope may consist of a single tube that incorporates a series of cuts (e.g., reliefs, slits, etc.) along its length to allow for improved flexibility as well as a desirable stiffness.

The bending section 103 may be designed to allow for bending in two or more degrees of freedom (e.g., articulation). A greater bending degree such as 180 and 270 degrees (or other articulation parameters for clinical indications) can be achieved by the unique structure of the bending section. In some cases, the bending section may be fabricated separately as a modular component and assembled to the insertion shaft. In some cases, the bending section may further incorporate minimalist features thereby reducing cost and increasing reliability. For example, the bending section may incorporate a cut pattern that beneficially allows for a greater degree of tube deflection to achieve a desired tip displacement relative to the insertion shaft.

FIG. 2 shows an example of a robotic endoscope system supported by a robotic support system. In some cases, the handle portion may be in electrical communication with the instrument driving mechanism (e.g., instrument driving mechanism 220) via an electrical interface (e.g., printed circuit board) so that image/video data and/or sensor data can be received by the communication module of the instrument driving mechanism and may be transmitted to other external devices/systems. As described elsewhere herein, the provided viewing direction adjustment method may beneficially reduce the bandwidth for the image data or improving the data rate.

In some embodiments, the electrical interface may also comprise optic interface or connector for illumination fibers of the illumination system. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

As shown in FIG. 2, a robotic endoscope 220 may comprise a handle portion 213 and a flexible elongate member 211. In some embodiments, the flexible elongate member 211 may comprise a shaft, steerable tip and a steerable section. The robotic endoscope 220 can be the same as the steerable catheter assembly as described in FIG. 1. The robotic endoscope may be a single-use robotic endoscope. In some cases, only the catheter may be disposable. In some cases, at least a portion of the catheter may be disposable. In some cases, the entire robotic endoscope may be released from the instrument driving mechanism and can be disposed of. The endoscope may contain varying levels of stiffness along its shaft, as to improve functional operation.

The robotic endoscope can be releasably coupled to an instrument driving mechanism 220. The instrument driving mechanism 220 may be mounted to the arm of the robotic support system or to any actuated support system as described elsewhere herein. The instrument driving mechanism may provide mechanical and electrical interface to the robotic endoscope 220. The mechanical interface may allow the robotic endoscope 220 to be releasably coupled to the instrument driving mechanism. For instance, the handle portion of the robotic endoscope can be attached to the instrument driving mechanism via quick install/release means, such as magnets and spring-loaded levels. In some cases, the robotic endoscope may be coupled or released from the instrument driving mechanism manually without using a tool.

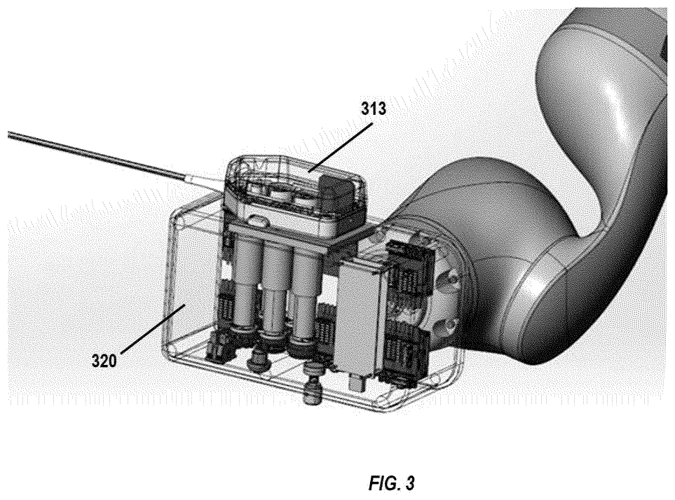

FIG. 3 shows an example of an instrument driving mechanism 320 providing mechanical interface to the handle portion 313 of the robotic endoscope. As shown in the example, the instrument driving mechanism 320 may comprise a set of motors that are actuated to rotationally drive a set of pull wires of the catheter. The handle portion 313 of the catheter assembly may be mounted onto the instrument drive mechanism so that its pulley assemblies are driven by the set of motors. The number of pulleys may vary based on the pull wire configurations. In some cases, one, two, three, four, or more pull wires may be utilized for articulating the catheter.

The handle portion may be designed allowing the robotic endoscope to be disposable at reduced cost. For instance, classic manual and robotic endoscope may have a cable in the proximal end of the endoscope handle. The cable often includes camera video cable, and other sensors fibers or cables such as electromagnetic (EM) sensors, or shape sensing fibers. Such complex cable can be expensive adding to the cost of the endoscope. The provided robotic endoscope may have an optimized design such that simplified structures and components can be employed while preserving the mechanical and electrical functionalities.

In some case, the handle portion may be housing or comprise components configured to process image data, provide power, or establish communication with other external devices. In some cases, the communication may be wireless communication. For example, the wireless communications may include Wi-Fi, radio communications, Bluetooth, IR communications, or other types of direct communications. Such wireless communication capability may allow the robotic bronchoscope function in a plug-and-play fashion and can be conveniently disposed after single use. In some cases, the handle portion may comprise circuitry elements such as power sources for powering the electronics (e.g., camera and LED light source) disposed within the robotic endoscope or catheter.

The handle portion may be designed in conjunction with the catheter such that cables can be reduced. For instance, the catheter portion may employ a design having working channel allowing instruments to pass through the robotic endoscope, low cost electronics such as a chip-on-tip camera, optics as part of the illumination system, and EM sensors located at optimal locations in accordance with the mechanical structure of the catheter. This may allow for a simplified design of the handle portion. For example, the handle portion may include a proximal board where the camera cable, optic fiber, and EM sensor cable terminate while the proximal board connects to the interface of the handle portion and establishes the electrical connections to the instrument driving mechanism. As described above, the instrument driving mechanism is attached to the robot arm (robotic support system) and provides a mechanical and electrical interface to the handle portion. This may advantageously improve the assembly and implementation efficiency as well as simplify the manufacturing process and cost. In some cases, the handle portion along with the catheter may be disposed of after a single use.

The catheter of the endoscope may include a lumen sized to receive a lumen or working channel to receive an instrument. Various instruments can be inserted through the lumen such as biopsy needle, graspers, scissors, baskets, snares, curette, laser fibers, stitching tools, balloons, morcellators, various implant or stent delivery devices, and the like.

The imaging device, the illumination components (e.g., DOE), EM sensor may be integrated to the distal tip of the catheter. For example, the distal portion of the catheter may comprise suitable structures matching at least a dimension of the above components. In some cases, the distal tip may have a dimension so that the one or more electronic components or optics can be embedded into the distal tip. For instance, the imaging device may be embedded into a cavity at the distal tip. The cavity may be integrally formed with the distal portion and may have a dimension matching a length/width of the camera such that the camera may not move relative to the distal tip.

The power to the camera may be provided by a wired cable. In some cases, the cable wire may be in wire bundle providing power to the camera as well as other circuitry at the distal tip of the catheter. The camera may be supplied with power from a power source disposed in the handle portion of the catheter via wires, copper wires, or via any other suitable means running through the length of the hybrid probe. In some cases, real-time images or video (of the tissue or organ) captured by the camera may be transmitted to external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system.

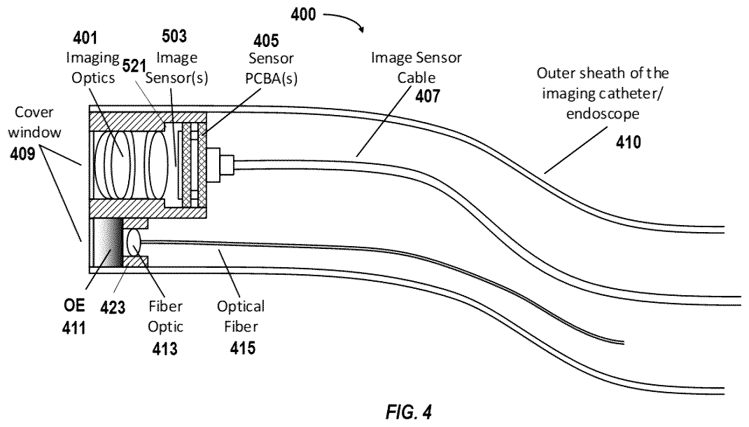

FIG. 4 shows an example of a configuration 400 of the distal portion of the system. In the illustrated example, the illumination system may comprise an optical component such as a diffractive optical element (DOE) 411 that directs the energy from the output end of the optic fiber 415 into a spatial distribution that may be desirable for the image sensor(s) that perform image acquisition. The DOE may shape the illumination light to better match the field of view of the imaging system. The optical element (OE) 411 may be located at the distal tip with an optic fiber 415. The optical element may be located next to a cover window 409 and integral to the distal tip. For example, the distal tip may comprise a structure receiving the optical element where glue may or may not be used. In some cases, the cover window 409 may be placed at the forwarding end face of the distal tip providing precise positioning of the DOE(s). In some cases, the cover window 409 may provide sufficient room for the glue of the DOE(s) and the cover 409 may be composed of transparent material matching the refractive index of the glue so that the illumination light may not be obstructed. In some cases, the cover window 409 may comprise a protection layer such as a thin layer of biocompatible glue applied to the front surface to provide protection while allowing light transmission.

The distal end of the optic fiber 415 may be terminated by a fiber optic 413 and fixed to the distal portion. In some cases, the fiber optic 413 may be configured to couple the focused mixed light into the center of the DOE 411 through the end face of the fiber optic at normal incidence.

The distal portion may also comprise an imaging device. The imaging device can be the same as the imaging device as described elsewhere herein. For example, the imaging device may comprise optical elements 401 and image sensor 403 for capturing image data. The imaging device may be capable of digitally adjusting a viewing direction. In some cases, power to the camera may be provided by a wired cable 407. In some cases, image and/or video data from the camera may be transmitted down the length of the catheter 410 to the processors situated in the handle portion via wires 407, copper wires, or via any other suitable means. In some cases, image or video data may be transmitted via the wireless communication component in the handle portion to an external device/system. In some cases, real-time images or video of the tissue or organ may be transmitted to external user interface or display wirelessly. The wireless communication may be WiFi, Bluetooth, RF communication or other forms of communication.

The imaging device may have parameters such as field of view, detection sensitivity and the like that may require a desirable illumination. As described above, the illumination system may be capable of adjusting the spatial distribution of the illumination based at least in part on the imaging device or image acquisition properties. In some cases, the illumination may be controlled to improve uniformity of one or more optical factors (e.g., color, contrast) across the imaging sensor.

The imaging device, the illumination components (e.g., DOE), or EM sensor may be integrated to the distal tip. As shown in the example, the distal portion may comprise suitable structures matching at least a dimension of the above components. The distal tip may have a structure to receive the camera, illumination components (e.g., DOE) and/or the location sensor. For example, the camera may be embedded into a cavity 421 at the distal tip of the catheter. The cavity may be integrally formed with the distal portion of the cavity and may have a dimension matching a length/width of the camera such that the camera may not move relative to the catheter. In some cases, the distal portion may comprise a structure 423 having a dimension matching a dimension of the DOE 411.

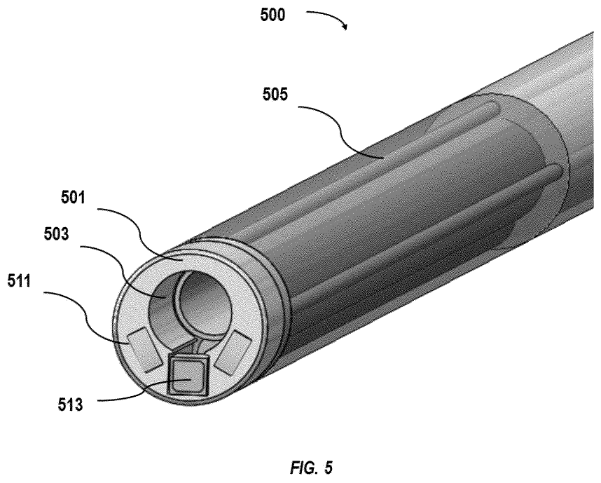

It should be noted that the illuminating system may include any suitable light sources such as LED and/or others. FIG. 5 shows another example of a distal tip 500 of an endoscope. In some cases, the distal portion or tip of the catheter 500 may be substantially flexible such that it can be steered into one or more directions (e.g., pitch, yaw).

The distal portion of the catheter may be steered by one or more pull wires 505. The distal portion of the catheter may be made of any suitable material such as co-polymers, polymers, metals or alloys such that it can be bent by the pull wires. In some embodiments, the proximal end or terminal end of one or more pull wires 505 may be coupled to a driving mechanism (e.g., gears, pulleys, capstan etc.) via the anchoring mechanism as described above.

The pull wire 505 may be a metallic wire, cable or thread, or it may be a polymeric wire, cable or thread. The pull wire 505 can also be made of natural or organic materials or fibers. The pull wire 505 can be any type of suitable wire, cable or thread capable of supporting various kinds of loads without deformation, significant deformation, or breakage. The distal end or portion of one or more pull wires 505 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The one or more electronic components may comprise an imaging device, illumination device or sensors. In some embodiments, the imaging device may be a video camera 513. The imaging device may comprise optical elements and image sensor for capturing image data.

The illumination device may comprise one or more light sources 511 positioned at the distal tip. The light source may be a light-emitting diode (LED), an organic LED (OLED), a quantum dot, or any other suitable light source. In some cases, the light source may be miniaturized LED for a compact design or Dual Tone Flash LED Lighting.

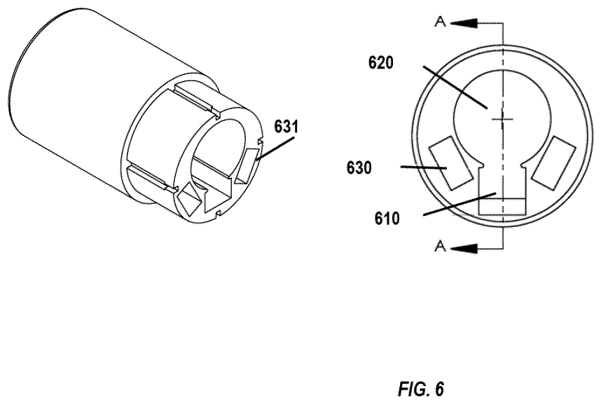

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 6 shows an example distal portion of the catheter with integrated imaging device and the illumination device. A camera may be located at the distal portion. The distal tip may have a structure to receive the camera, illumination device and/or the location sensor. For example, the camera may be embedded into a cavity 610 at the distal tip of the catheter. The cavity 610 may be integrally formed with the distal portion of the cavity and may have a dimension matching a length/width of the camera such that the camera may not move relative to the catheter. The camera may be adjacent to the working channel 620 of the catheter to provide near field view of the tissue or the organs. In some cases, the attitude or orientation of the imaging device may be controlled by controlling a rotational movement (e.g., roll) of the catheter. In some cases, the mechanical/physical control of the orientation of the camera may be combined with a digital adjustment of the viewing direction to achieve a desired viewing direction.

In some embodiments of the disclosure, miniaturized LED lights may be employed and embedded into the distal portion of the catheter to reduce the design complexity. In some cases, the distal portion may comprise a structure 530 having a dimension matching a dimension of the miniaturized LED light source. As shown in the illustrated example, two cavities 530 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiment, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover 531 may be placed at the forwarding end face of the distal tip providing precise positioning of the LEDs as well as sufficient room for the glue. The cover 531 may be composed of transparent material matching the refractive index of the glue so that the illumination light may not be obstructed.

### Digital Adjustment of Viewing Direction

In an aspect, methods and systems are provided for digitally adjusting a viewing direction of the vision system. In some embodiments, a viewing direction may be digitally adjusted by selecting a partial readout of an imaging sensor corresponding to a viewing direction/angle. For instance, the system and method herein may receive a command indicating a desired viewing direction (e.g., tilt angle) and may determine a region (e.g., addresses of pixels) in a sensor array corresponding to the desired viewing direction for outputting sensor readout. This beneficially allows for processing/outputting only partial or portion of a full sensor array readout thereby reducing the power consumption of the vision system. In some cases, high frame readout may be performed in the selected region such that bandwidth and power consumption can be reduced.

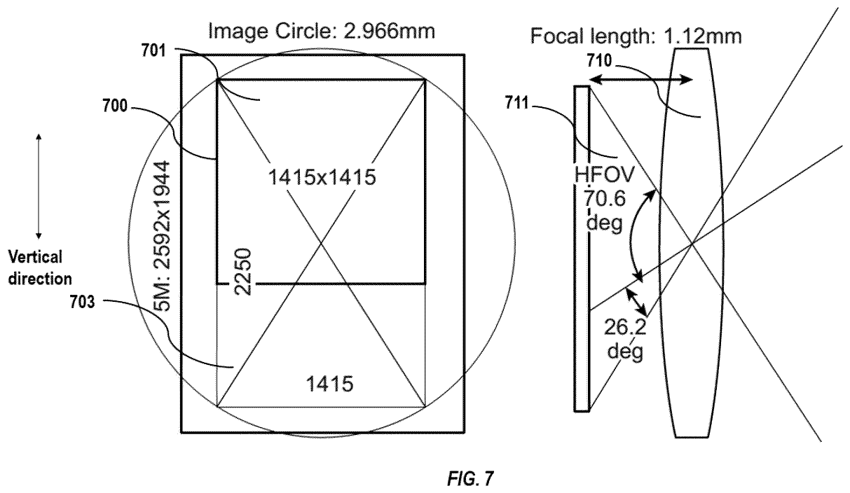

FIG. 7 schematically illustrates a method of performing digital tilt of a vision system. In some embodiments, the vision system may comprise an imaging device. The imaging device can be the same as described above. For example, the imaging device may be a camera comprising imaging optics (e.g., lens elements) 710 and image sensor (e.g., CMOS or CCD) 700. The image circle is the maximum sensor area that the lens can support. The image sensor may have any suitable size and/or aspect ratio. For example, the image sensor 700 may have a rectangular photo sensitive area with 16×9, 11:8, 19:9, 35:18, 9:5, 8:3, 7:3, 4:3, 3:2 aspect ratio (length:height) and any other ratios.

The viewing direction may be adjusted in one or more directions. For example, by arranging a direction of the image sensor 700 and selection of a region-of-Interest (ROI) 700, the viewing direction may be adjusted along a vertical direction (e.g., tilt angle) or horizontal direction (e.g., pan angle), or a combination of both. In the illustrated example, the image sensor 700 may be arranged such that the length (long edge) is along the vertical direction, and a ROI 701 may be selected (e.g., shifted in the vertical direction) corresponding to an angle/field of view 711 adjusted in the vertical direction (e.g., tilt angle). It should be noted that the image sensor 700 can be arranged in any direction depending on the use application.

In some cases, only signals from the region-of-Interest (ROI) 701 out of entire image sensor 700 may be read out and processed to generate an image. The ROI 701 may be a portion of the entire image sensor 700 while signals from at least a portion of the image sensor 703 is not outputted for processing. By generating only some parts of an entire image, required bandwidth and power consumption are saved.

The ROI 701 can have any suitable aspect ratio depending on the use application or user preference. For example, for viewing a substantially circular or tubular tunnel (e.g., colon, trachea/bronchi, esophagus), a ROI with 1:1 (or 4:3 or 5:4 or other) aspect ratio may be selected from the entire image sensor. For instance, a colonoscope may have a 5:4 aspect ratio and a ROI with a 5:4 block may be selected. In the illustrated example, the ROI 701 may have a size/dimension that is within the image circle supported by a field/angle of view of the optical system 710 (e.g., diagonal field of view of 90 degree). In some cases, the ROI may be a fixed-sized block (e.g., 1415×1415). Alternatively, the ROI may have variable size. In the example, the horizontal field of view (HFOV) and the diagonal field of view (DFOV) may determine a range of the digital tilt. For example, a HFOV of 70.6 degree may determine that a digital tilt range of 26.2 degree (90-70.6 degree).

In some embodiments, the viewing angle adjustment (e.g., digital tilt) may be performed by electronically shifting the ROI 701 in one or more directions (e.g., up-down, left-right) within the sensor area 700. The ROI may be determined by external logics. As described above, the ROI may be pixel array with fixed block size (e.g., 1415×1415 pixels). Alternatively, the block size may be variable by the external control.

In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor and circuitry to combine or serialize the data so that it can be transmitted in a minimum number of electrical conductors. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera.

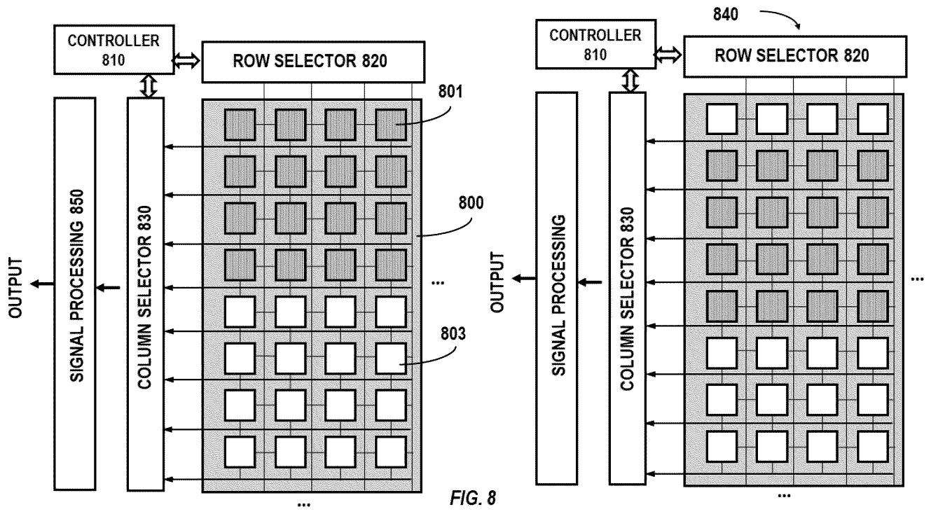

FIG. 8 shows an example of complementary metal oxide semiconductor (CMOS) image sensor with digitally viewing direction adjustment capability. The COMS image sensor may comprise a detector array (e.g., array of photodiodes) 800. In some cases, the CMOS sensor may be passive pixel sensor that photocurrent generated from incident light is converted to voltage in the column-parallel charge amplifier. Alternatively, the CMOS sensor may have an active pixel sensor architecture that each pixel has an in-pixel amplifier.

In the illustrated example, a ROI may comprise array of pixels 801 that may be selected by the row selector 820 and/or the column selector 830 (depending on how the image sensor is arranged). For example, the column selector 830 may comprise selection transistors to select addresses of photodiodes in a ROI for the sensor read out (e.g., starting column, number of columns). By shifting the starting column number as shown in the example 840, a different region may be selected that may correspond to a different viewing direction. The photodiodes 801, 803 in the sensor array 800 may be individually addressable. Alternatively, the photodiodes 801, 803 in the sensor array 800 may be addressable by row or column. The controller unit 810 may be in communication with row selector 820 and column selector 830 by generating row/column signals to select an active region for outputting the sensor signals based on a viewing direction (e.g., tilt angle). The output signals produced by a photosensor may be transmitted to a processing circuit 850 to generate a pixel value (e.g., amplitude).

As described above, the provided method may beneficially reduce the power consumption by reducing the data rate and/or bandwidth. Below shows an example of the power consumption between a full sensor read out and partial sensor read out.

| 1. Mode | 2. Resolution | 3. Data rate | 4. Power consumption |
|---|---|---|---|
| 5. Full read out | 6. 2592 × 1944 | 7. 1.51 Gbps | 8. 96 mW |
| 9. Partial read out | 10. 1415 × 1415 | 11. 0.60 Gbps | 12. 67 mW |

In addition to the power consumption, the memory consumption is also reduced with the decrease of the output image size (e.g., image size reduction is 60%).

In alternatively embodiments, instead of selecting the region for outputting the sensor signals, photodiodes 801 in an active region in the detector array may be enabled and photodiodes 803 in inactive region may be disabled. This may further reduce the power consumption of the sensor. For instance, each photosensor may be connected with a pixel level circuit, allowing for individual control of respective photosensor. For example, depending on the type of photodiode, a photodiode may be disabled or powered-off by lowering a bias voltage below breakdown such as through a control switch of the pixel-level circuit. In some cases, the controller unit 810 may be in communication with the row selector 820 and column selector 830 comprising array of driver transistors that may be individually addressable via column signals and row signals generated by the controller unit 810. For example, the driver transistors of the column selector 830 may be individually activated (e.g., biased so as to be conducting) so as to vary power/current provided to a respective one or more photodiodes 801 in the active region thereby enabling the selected photodiodes 831 or disabling the photodiodes 803.

The controller unit 810 may receive a command indicating a viewing direction such as tilt or pan angle (with respect to the axial direction of the endoluminal device, a global reference and other reference frame). The controller unit 810 may determine a ROI depending on the viewing direction, then generate row signals (e.g., starting row, number of rows) to the row selector 820 and/or column signals (e.g., starting column, number of columns) to the column selector 830. In some cases, the ROI may be determined based on a mapping relationship between a viewing direction (e.g., tilt angle) and a ROI (e.g., defined by address and size). The mapping relationship may be predetermined such as obtained during a calibration process. For example, a look up table for a tilt angle may be predetermined and stored in a memory that is accessible by the controller unit 810.

The sensor output signals (e.g., voltage signals) may be processed by the signal processing circuit 850 to generate an output image. The signal processing circuit may receive readout from the detector array and perform signal processing. In some cases, the controller unit 810 and/or the signal processing circuit 850 may be assembled on the same substrate with the detector array (e.g., using CMOS) technology or be connected to the detector array. For example, the controller unit 810 and/or the signal processing circuit 850 may be an integrated circuit such as field programmable gate arrays (FPGAs), application specific integrated circuit (ASIC) or digital signal processors (DSPs). Alternatively, the controller unit 810 may not be fabricated on the same substrate with the detector array and may be in communication with the detector array.

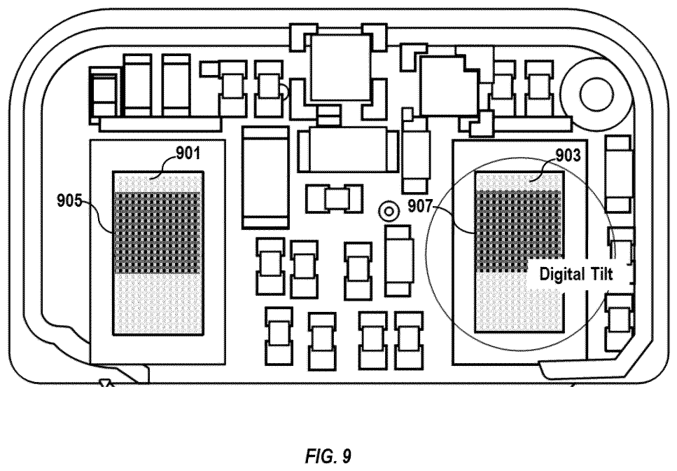

In some embodiments, the imaging system may be stereoscopic imaging system including at least two image sensors. FIG. 9 shows an example of a stereo camera system with digital tilt capabilities. In the illustrated example, two image sensors 901, 903 may be arranged in a stereo manner (side-by-side) and the ROI in each image sensor 905, 907 may be selected for the sensor output corresponding to a viewing direction (e.g., tilt angle) as described above. In some cases, when the two image sensors 901, 903 are not perfectly aligned horizontally, an offset between the two image sensors in the vertical direction may be calculated for applying the digital tilt so that the two ROI 905, 907 displayed to a user are aligned. In some cases, different sensing regions in the two of the imaging sensors are selected to align the different sensing regions in a selected direction (e.g., vertical direction). For instance, the offset in the vertical direction may be translated to offset of row address between the two image sensors such that the ROI in each image sensor 905, 907 is aligned. This beneficially allows for convenient alignment of field of view of the two image sensors in a direction (e.g., horizontal direction) without perfect physical alignment of the two image sensors. In some cases, the offset may be obtained from a calibration process and may be stored in a look up table. The controller unit (e.g., controller unit 810) may determine the row and/or column address for each image sensor based at least in part on the offset.

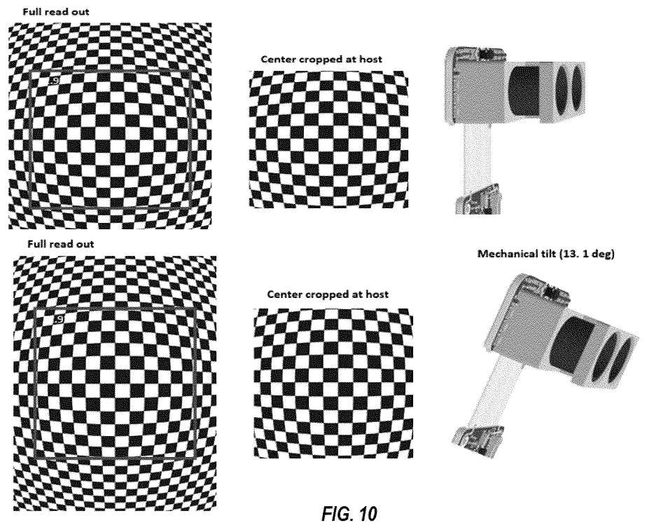

As described above, the image quality across a full image sensor may not be uniform due to the optical system physical characteristics. For example, optical factors such as distortion, contrast (e.g., modulation transfer function (MTF)), color and the like may not be uniform across the entire sensor. FIG. 10 shows an example of distortion across an entire image frame/full sensor readout. As illustrated in the example, the distortion effect is not uniform between a center region of the image frame and an off-center region. When mechanical tilt is performed, the distortion is uniform because the center region of the image frame is cropped for the output.

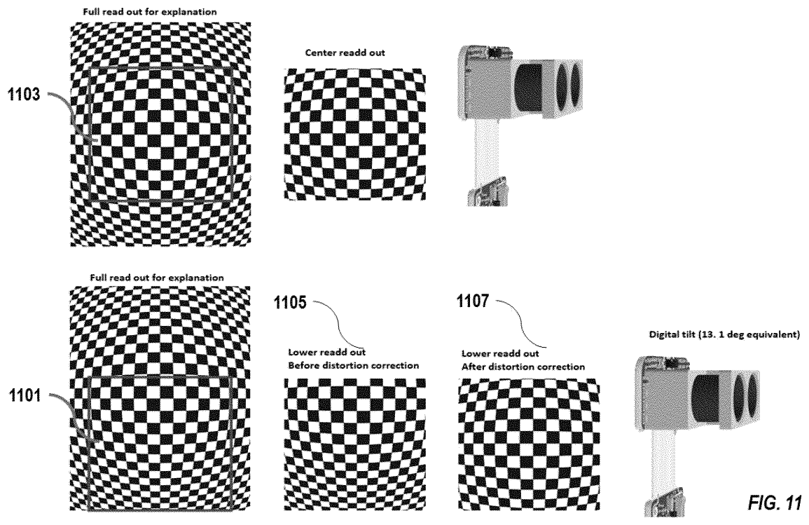

The method and system herein may provide a uniform image quality regardless which portion/region of the image sensor is selected. For example, the partial readout may be processed such that the one or more optical parameters (e.g., MTF, distortion, color, etc.) of an output image may be substantially uniform regardless the digital viewing direction. In some cases, the output image may be processed to account for one or more optical factors thereby providing uniform image quality. In some cases, the sensor readout may be further processed to generate an image with an image quality same as performing a mechanical tilt of the camera. As shown in FIG. 11, a ROI 1101 corresponding to a tilt angle (e.g., 13.1 degree) may have distortion different from that of the center region 1103. An image transformation may be applied to image 1105 generated from the ROI 1101 readout and the final output image 1107 may have the same distortion effect as the center region 1103 (as if a mechanical tilt was applied). Similarly, other optical factors such as color, contrast (MTF) may also be corrected or accounted for by performing imaging processing to the image. In some cases, the characteristics or the optical factors across the image sensor may be obtained during a calibration process.

In some cases, image transformation may be applied to correct the projective distortion, the color distortion or contrast. Any suitable computer vision and image processing methods may be utilized to correct the one or more optical factors. For example, the projective transformation may be locally approximated by a similarity or affine transformation with some scale or affine invariants or other methods such as transform invariant low-rank textures (TILT). The output image may be processed by applying the image transformation using one or more processors. In some cases, the one or more processors may include the signal processing assembled on the same substrate with the detector array (e.g., using CMOS) technology or be connected to the detector array. Alternatively, the one or more processors may be in communication with the COMS sensor and may be placed at any location of the system. For example, the image processing may be performed by the processor located at the handle portion of the system as described above.

In some cases, camera calibration may be performed to obtain a mapping relationship between the ROI and a viewing direction (e.g., tilt angle), an offset between two image sensors in stereoscopic imaging, the one of more optical factors and/or one or more intrinsic parameters of the camera (e.g., focal length, principal point, lens distortion, etc.). The camera calibration process can use any suitable method. For example, recognizable patterns (e.g., checkerboards) with known or unknown locations/orientations relative to the camera may be used to determine the distortion. The camera may be positioned into a variety of different point of views with respect to the patterns and/or the pattern may be positioned into different positions/orientations with respect to the camera. The process may be repeated multiple times on the same pattern. In some cases, the process may be repeated using different patterns.

In some embodiments, the digital tilt angle (pan or other angle) may be combined with mechanical tilt angle allowing for flexibility in adjusting an angle of view and projection angle. For example, a mechanical angle offset may be applied to the image sensor module mount to bias the tilt range to top or bottom. For instance, for a camera lens and sensor arrangement that provide theta degrees of digital tilt angle range (e.g., theta degree up and down from the center), the camera may be tilted mechanically downwards at an angle (e.g., theta angle) with respect to the axial direction of the tip of the endoscope so that the uppermost tilt angle is looking straight ahead at 0 degrees, and the bottommost tilt angle is tilted downwards at two times of the theta degrees. In another example, upon receiving a command to tilt the view direction of the camera by a certain degree, the system may automatically decompose the tilt angle by determining a first fraction of the angle achieved via mechanical tilt of the camera (e.g., articulate the camera upwards by the first fraction) and a second fraction of the angle achieved via the digital tilt (e.g., determining a partial readout of the sensor corresponding to the second fraction of the angle). For example, a first fraction or a first angle is used to determine the sensing region on the imaging sensor and a second fraction or a second angle is used to command an adjustment of an angle of the imaging sensor. In some cases, the determination of the first fraction of the angle achieved via a mechanical tilt of the camera may be based at least in part on a current physical orientation of the tip of the endoscope. For example, if the catheter distal portion is already bent to a higher degree (e.g., determined based on the EM sensor data), a greater fraction of the tilt angle may be allocated to the digital tilt to achieve a desired viewing direction.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for controlling a vision system for an articulatable medical device, the method comprising:
receiving a command indicative of adjusting a viewing direction of the articulatable medical device;
determining a sensing region on an imaging sensor based at least in part on the viewing direction;
generating an image based on signals from the sensing region; and
processing the image to correct at least one of a projection distortion, a contrast and a color of the image.

2. The method of claim 1, wherein the viewing direction comprises a tilt angle.

3. The method of claim 2, wherein the sensing region is a portion of the imaging sensor and is selected in a vertical direction.

4. The method of claim 1, wherein the signals are read out from the sensing region via a row selector and a column selector.

5. The method of claim 1, wherein an array of photodiodes are enabled upon determining the sensor region and wherein photodiodes not in the sensing region are disabled.

6. The method of claim 1, wherein the sensing region is determined based on the viewing direction and a predetermined mapping relationship.

7. The method of claim 1, wherein the vision system comprises two of the imaging sensors providing stereoscopic imaging.

8. The method of claim 7, wherein different sensing regions in the two of the imaging sensors are selected to align the different sensing regions in a vertical direction.

9. The method of claim 1, wherein the projection distortion, the contrast or the color of the image is corrected by applying image transformation.

10. The method of claim 1, further comprising decomposing the viewing direction of the articulatable medical device into a first angle and a second angle.

11. The method of claim 10, wherein the first angle is used to determine the sensing region on the imaging sensor and the second angle is used to control an orientation of the imaging sensor.

12. The method of claim 1, wherein the imaging sensor is located at a distal end of the articulatable medical device.

13. A non-transitory computer-readable storage medium including instructions that, when executed by one or more processors, cause the one or more processors to perform operations comprising:
receiving a command indicative of adjusting a viewing direction of the articulatable medical device;
determining a sensing region on an imaging sensor based at least in part on the viewing direction;
generating an image based on signals from the sensing region; and
processing the image to correct at least one of a projection distortion, a contrast and a color of the image

14. The non-transitory computer-readable storage medium of claim 13, wherein the viewing direction comprises a tilt angle.

15. The non-transitory computer-readable storage medium of claim 14, wherein the sensing region is a portion of the imaging sensor and is selected in a vertical direction.

16. The non-transitory computer-readable storage medium of claim 13, wherein the signals is read out from the sensing region via a row selector and a column selector.

17. The non-transitory computer-readable storage medium of claim 13, wherein an array of photodiodes are enabled upon determining the sensor region and wherein photodiodes not in the sensing region are disabled.

18. The non-transitory computer-readable storage medium of claim 13, wherein the sensing region is determined based on the viewing direction and a predetermined mapping relationship.

19. The non-transitory computer-readable storage medium of claim 13, wherein the vision system comprises two of the imaging sensors providing stereoscopic imaging.

20. The non-transitory computer-readable storage medium of claim 19, wherein different sensing regions in the two of the imaging sensors are selected to align the different sensing regions in a vertical direction.
